(19) United States
(12) Patent Application Publication
(10) Pub. No.: US 2026/0223954 A1
(43) Pub. Date: Aug. 6, 2026

(54) Temperature Management System

(71) Applicant: TEMPCHEM LLC, Buffalo, NY (US)

(72) Inventor: Samuel L. MORRIS, III, Buffalo, NY (US)

(21) Appl. No.: 19/530,456

(22) Filed: Feb. 5, 2026

Related U.S. Application Data
(60) Provisional application No. 63/754,262, filed on Feb. 5, 2025.

(51) Int. Cl. A24F 40/57; A24F 40/51; A24F 40/65; A61M 15/00; G01K 13/00

(52) U.S. Cl. CPC A24F 40/57; A24F 40/51; A24F 40/65; A61M 15/0065; G01K 13/00

(57) ABSTRACT

A device, a system, and method of using a smoke substrate. The invention confirms that the smoke substrate is authentic and used at a best temperature index to maximize the alleged smoke substrate medicinal and/or alternative benefits.

This application claims priority to U.S. provisional application Ser. No. 63/754,262, which was filed on Feb. 5, 2025.

### BACKGROUND OF THE INVENTION

### Applicant's Prior Patented Product

Applicant is currently the owner of U.S. Pat. No. 11,740,133 for a Temperature Sensing System. In that patent, it was expressed that in many systems, it is important to accurately measure the temperature of a target material. For example, a system for vaporizing such materials as botanical extracts includes a receptacle, commonly referred to as a banger, for holding the botanical extracts while it is vaporized such as with a butane torch. If the target material is under heated, inadequate vaporization can occur. If it is overheated, burning can occur.

Accordingly, there is a need for a temperature sensing device that accurately measures the temperature of a heated targeted material.

The invention is directed to a temperature sensing device having a housing that includes a display, and an extension extending from the housing by at least 1.5 inches. The extension has a proximal section at the housing and an opposed distal section, the distal section being movable relative to the housing so that it can be brought near an appropriate location proximate to the target material. A temperature sensor is at the distal section of the extension for sensing the temperature of a target material. The target material can provide a vapor when heated to a selected temperature. The temperature sensor provides an output related to the temperature of the target material. There is a connector from the sensor to the housing for transmitting the output of the temperature sensor to the housing for display.

The device can have a light source at the distal section of the extension for aiming the sensor at the target material.

Optionally, the device has a thermal insulator at the distal section of the extension for protecting the temperature sensor from heat from the target material. The thermal insulator can have a first hole in the thermal insulator so that the temperature sensor is not covered by the thermal insulator. Optionally, the thermal insulator can have a second hole in the thermal insulator so that the light source is not covered by the thermal insulator.

The display can show the temperature sensed by the temperature sensor.

The device can further comprise a conversion circuit in the housing for converting the output of the temperature sensor to the displayed temperature.

The housing can have a selector key for inputting to the conversion circuitry the container material constituted by a container containing the target material for adjusting the displayed temperature based on the infrared transmissivity of the container material.

The housing can also have an input key for setting a target temperature of the target material and an alert generator for generating an alert when the target temperature is sensed by the temperature sensor.

The housing can also have a selector key for the type of material of a container containing the target material.

The housing can comprise a recess for receiving a banger cap.

The extension can be a flexible tube with the connector therein. Optionally, the extension is removable. Optionally, the device further comprises a replacement extension for changing out the removable extension.

Optionally, the device has memory for tracking the number of times the device has sensed the target temperature, and optionally, for storing how much time it takes to reach the target temperature.

The device can be used by the steps of: a) placing the target material into a container that is translucent or transparent; b) heating the target material to a temperature that generates a vapor from the target material; and c) detecting the temperature of the target material with the device by moving the extension relative to the housing to a location proximate to the container.

The target material can be cannabis, and the step of heating can comprise heating the cannabis to a temperature between 230 and 330 degrees Centigrade.

When the device has a light source generating a light beam at the distal section of the extension for aiming the sensor at the target material, the method can comprise aiming the beam generated at the target material.

With reference to FIGS. 1-11, a temperature sensing device 200 is shown. The temperature sensing device 200 has a recess 202 for receiving a banger cap 204. The banger cap 204 is a dabbing accessory that restricts airflow and traps heat around a domeless nail or banger (a container or a thermal conducting device) 205, allowing a smoke substrate, such as a cannabis concentrate, 207, to vaporize at a lower temperature, preserving the quality and flavor of the concentrate. With reference to FIG. 11, use of the temperature sensing device 200 is shown relative to a standard vaporizing rig 300 having the container (banger or thermal conducting device) 205 for holding the smoke substrate 207 to be heated. The banger cap 204 is placed on the banger (container or thermal conducting device) 205 when a user is inhaling vapor from the heated smoke substrate 207 and helps regulate airflow into the device 200.

Table 1, principally with regard to FIG. 8, presents the components of the device 200:

TABLE 1

| ITEM NO. | QTY. | DESCRIPTION |
|---|---|---|
| 100 | 1 | HOUSING BASE |
| 101 | 1 | HOUSING COVER |
| 102 | 1 | SENSOR BARREL LEFT |
| 103 | 1 | SENSOR BARREL RIGHT |
| 104 | 1 | BARREL CAP TOP |
| 105 | 1 | BARREL CAP BOTTOM |
| 106 | 1 | SILICONE INSERT |
| 107 | 1 | SILICONE BARREL COVER |
| 108 | 1 | BASE WEIGHT |
| 109 | 1 | PCB WITH A WEIGHT BRACKET |
| 110 | 1 | DISPLAY BRACKET |
| 111 | 1 | BARREL FLEXLINE |
| 112 | 1 | USB PORT |
| 113 | 1 | USER BUTTONS |
| 114 | 3 | PCB CONTACTS FOR BUTTONS |
| 115 | 1 | MAIN PCB (printed circuit board) |
| 116 | 1 | SENSOR PCB (printed circuit board) |
| 117 | 1 | OLED DISPLAY |
| 118 | 1 | BATTERY |
| 119 | 1 | CABLE FROM PCB TO SENSOR |
| 120 | 1 | BARREL LED |
| 121 | 1 | TEMPERATURE SENSOR |
| 122 | 1 | SENSOR TUBE |
| 123 | 1 | SENSOR COLLAR |
| 124 | 1 | PCB MOUNT SPEAKER |
| 125 | 2 | LOCKING NUT |
| 126 | 8 | TORX SELF TAPPING SCREWS |
| 127 | 4 | ANTI SKID FOOT |
| 128 | 2 | HOLES IN INSULATING COVER |
| 129 | 1 | SENSOR ASSEMBLY |
| 130 | 1 | SENSOR BARREL |
| 131 | 1 | MICROPROCESSOR |

The device 200 comprises a housing 206 formed of a housing base 100 and a housing cover 101 with a base weight 108 held in place by a weight bracket combined with a printed circuit board (PCB) 109 so the housing stays in position during use. The bottom of the housing base 100 has antiskid feet 127. The housing cover 101 has an opening for an OLED (organic light emitting diode) display 117 held in place by a bracket 110. The display 117 displays information from the device 200, including but not limited to battery level, a light alert indicator, a sound alert indicator, an LED guide indicator, temperature reading and degree scale information, and banger type. The LED guide indicator assists the user in aligning the [temperature] sensor 121 with the banger 205.

The printed circuit board 109 controls the main functions of the device 200, creates (a) the output from [the temperature] sensor 121 and (b) alerts the sound and lights to flash and ding upon set parameters. Also, within the housing 206 is a circuit board 115 that houses a microprocessor 131 for running the functions of the entire [device 200]. The housing 206 includes a PCB mount speaker 124 for sounding the sound alerts.

Contacts 114 are provided for the user buttons 113 to provide signals to the main printed circuit board 115. User button 113 project upward through the housing cover 101. Preferably, there are three user buttons 113, as best seen [at] FIG. 9. The two upper buttons 113A, 113B permit the user to scroll left and right through the menu. The bottom centrally located button 113C is the power/menu/set button. The user buttons 113 can be made from rigid or flexible plastic material or metal.

The housing cover 101 also includes a USB port 112 and a battery 118 such as a rechargeable lithium battery. The battery [118] can be charged using the USB port 112. Optionally the device 200 can be provided with a socket for external power or in place of or as an option to the battery 118. Components of the housing 206 are held together with Torx (trademark) tapping screws 126; however, other types of fasteners known in the art may be used.

The device 200 includes a temperature sensor 121, namely an infrared sensor. The appropriate temperature sensor depends on the application for which the device is used. For example, if used for heating cannabis, the temperature sensor 121 preferably is effective between 230° C.-330° C. and a preferred sensor range is 93-425° C.

A preferred temperature sensor 121 is a Heimann thermopile sensor. The Heimann thermopile sensor is preferred because it comprises a CMOS compatible sensor chip plus a thermistor reference chip, good sensitivity, a small temperature coefficient of sensitivity, and high reproducibility and reliability.

The [temperature] sensor 121 has a storage temperature (meaning the temperature at which the sensor 121 can safely be stored at) of between −40° C. to 100° C., and an operating temperature (meaning the temperature at which the [temperature] sensor 121 can safely operate at) of between −20° C. to 100° C. Table 2 outlines the general and electrical parameters of the Heimann thermopile sensor 121:

TABLE 2
Limits

| Parameter | Symbol | Min | Typ. | Max | Units | Conditions |
|---|---|---|---|---|---|---|
| element | | | 1.2*1.2 | | mm² | absorbing area |
| size | | | | | | |
| field of | | | 16°@50% | | degree | Full FOV vs indicated Sv level |
| view | | | | | | |
| (FOV) | | | | | | |
| | | | 30°@10% | | | Diagonal cut |
| | | | 34°@5% | | | Tested with point source |
| resistance | R_{TP} | 69 | 84 | 112 | kΩ | −4° C. to 100° C. |
| signal | V_{S} | | 890 | | μV | T_{amb} = 25° C., T_{BB} = 100° C., 4.5 HZ, |
| voltage | | | | | | distance 15 mm |
| time | τ | | 10 | 13 | ms | |
| constant | | | | | | |
| noise | V_{RMS} | | 37 | | nV/√Hz | r.m.s., 25° C. |
| voltage | | | | | | |
| pin | Rᵢₛₒ | 30 | | | GΩ | 25° C. 50% RH, 11 V, DC |
| insulation | | | | | | |
| resistance | | | | | | |

Tables 3A and 3B outline the general and electrical parameters of the thermistor of the thermopile Heimann sensor 121:

TABLE 3A
Type Thermistor 100 kΩ
Limits

| Parameter | Symbol | Min | Typ. | Max | Units | Conditions |
|---|---|---|---|---|---|---|
| resistance | R_{TH} | 95 | 100 | 105 | kΩ | 25° C. |
| BETA-value | β | 3900 | 3940 | 3980 | K | 25° C./50° C. |

TABLE 3B

| T/° C. | Rth_min/Ohm | Rth_nom/Ohm | Rth_max/Ohm |
|---|---|---|---|
| −30 | 1557900 | 1655000 | 1753100 |
| −25 | 1163320 | 1234000 | 1306680 |
| −20 | 875826 | 928700 | 981974 |
| −15 | 665010 | 704500 | 744190 |
| −10 | 508730 | 538500 | 568370 |
| −5 | 392108 | 414600 | 437292 |
| 0 | 304466 | 321700 | 338934 |
| 5 | 238072 | 251400 | 264728 |
| 10 | 187444 | 197800 | 208056 |
| 15 | 148568 | 156600 | 164632 |
| 20 | 118404 | 124800 | 131096 |
| 25 | 95000 | 100000 | 105000 |
| 30 | 76537 | 80630 | 84713 |
| 35 | 62032 | 65380 | 68738 |
| 40 | 50543 | 53310 | 56077 |
| 45 | 41386 | 43680 | 45984 |
| 50 | 34070 | 35980 | 37890 |
| 55 | 28174 | 29770 | 31366 |
| 60 | 23405 | 24750 | 26095 |
| 65 | 19536 | 20670 | 21804 |
| 70 | 16383 | 17340 | 18297 |
| 75 | 13788 | 14600 | 15422 |
| 80 | 11653 | 12350 | 13047 |
| 85 | 9890 | 10480 | 11080 |
| 90 | 8421 | 8930 | 9444 |
| 95 | 7197 | 7635 | 8076 |
| 100 | 8172 | 6551 | 6935 |

Table 4 outlines the filter coating characteristic and lens parameters of the thermopile Heimann sensor 121:

TABLE 4
Filter F5.5
Limits

| Parameter | Min | Typ. | Max | Units | Conditions |
|---|---|---|---|---|---|
| average | 75 | | | % | 7.5 μm to 13.5 μm |
| transmission | | | | | |
| absolute | | | 1 | % | visual to 5 μm |
| transmission | | | | | |
| cut on at 5% | 5.2 | 5.5 | 5.8 | μm | 25° C. |
| back focal | | 3.0 | | mm | |
| length of lens | | | | | |

It is desirable that the temperature sensor 121 is proximate to a heated smoke substrate 207. For this purpose, the device 200 has an extension 220 that is at least 1.5 inches long and extends from the housing 206, where the extension 220 has a proximal section 222 at the housing 206 and an opposed distal section 224, wherein the distal section 224 is movable relative to the housing 206. The temperature sensor 121 is at the distal section 224 of the extension 220 for sensing the temperature of the smoke substrate 207 and providing an output related to the temperature of the smoke substrate 207. This is best seen in FIGS. 5 and 8. The extension 220 can be from about 3 to about 12 inches long so that the temperature sensor 121 is at least 1.5 inches and up to 12 inches, and preferably 7 inches, from the housing 206. Preferably, the extension 220 is a flexible tube 111.

A sensor assembly 129, which is best seen in FIG. 8, includes the temperature sensor 121, a sensor tube 122 and a collar 123, with components held in place with locking nuts 125. The sensor assembly 129 also includes a sensor barrel 130 formed of wall components 102 and 103, a barrel cap top portion 104 and a barrel cap bottom portion 105. Wall components 102 and 103 form two halves of the sensor barrel 130 that when coupled together, holding the remaining components of the sensor assembly 129, discussed in more detail below, therein. The sensor assembly 129 is located at the distal section 224 of the extension 220.

Output from the temperature sensor 121 is related to the temperature of the smoke substrate 207 and is transmitted to a sensor printed circuit board 116 via a cable 119 for transmission to the main sensor board 115.

The housing 206 includes a thermal insulating insert 106 and the sensor assembly 129 further includes a cover 107, wherein both the insulating insert 106 and the cover 107 are preferably made of silicone. This is for protecting the temperature sensor 121 from heat [generated] from the smoke substrate 207. The cover 107 is removable and remains on the sensor assembly 129 (gripping an exterior surface of the wall components 102, 103) via friction.

Preferably there is a light source such as a light emitting diode 120 as part of the sensor assembly 129. This can be used for aiming the sensor 121 at the smoke substrate 207 to be sure to get an accurate reading of the temperature of the smoke substrate 207.

Preferably the thermal insulating cover 107 has openings 128 for both the temperature sensor 121 and the light source 120 so they are not covered by the insulating material 107.

The housing 206 and other components can be formed of a plastic material such as ABS or nylon. Silicone can be used as the insulating material. Also, silicone can be used as a cushion for the recess 202. Recess 202 is configured to hold the banger cap 204 when the banger cap 204 is not in use. Silicone is desirable in recess 202 because the banger cap 204 can get hot and sticky during use. Accordingly, the silicone lined recess 202 provides a resting place for the banger cap 204 when not in use that can handle both the heat and the stickiness of the used banger cap 204.

FIG. 9 shows a flow chart for programming the device 200 utilizing the user buttons 113. The user buttons 113 are used to first power on the device 200, after which the main display screen 117 is shown. The user can then use the buttons 113 to set a target temperature and set an alert, such as a sound alert or a light alert or both so a user knows when the target temperature has been achieved. The alert indicates that heating the smoke substrate 207 should stop. The user then selects the banger type, completing the set-up and start-up of the device 200.

The user then lines up the sensor 121 with the banger 205. As noted above, the sensor assembly 129 includes an LED. When the LED is on, it provides a guiding beam of light. The beam should align with the edge of the banger 205 so that the temperature sensor 121 is centered under the hanger 205.

The main circuit board 115 converts the output of the temperature sensor 121 to the display temperature.

The device 200 can be provided with memory such as ram, rom, and flash memory. The memory can be used for tracking the number of times the device 200 has sensed the target temperature. Memory can also be used for storing how much time it takes to reach the target temperature.

A feature of the present invention is an ability to adjust the output for the different type of container being used. As referred to in FIG. 9 there is an option “select banger type”. For example, typical containers for the target material can be made of quartz or opaque quartz. The infrared transmissivity of these two types of materials is different, and thus by using a user key referred to as a selector key, the type of material holding the smoke substrate 207 can be inputted into the main circuit board 115 for adjusting the display temperature based on the infrared transmissivity of the container material.

Referring now to FIGS. 10A-10M, there are shown circuit diagrams for the circuits used in the device 200.

FIG. 10A shows a circuit for power management.

FIG. 10B shows a circuit for a VCC power supply.

FIGS. 10C and 10D show circuits for a sensor and peripheral compensation circuit.

FIG. 10E shows a circuit for the MCU.

FIG. 10F shows a circuit for communication isolation resistance.

FIG. 10G shows the circuit for an MCU program burning interface.

FIG. 10H shows a circuit for a display driver chip.

FIG. 10I shows a circuit for a flash memory chip.

FIG. 10J shows a circuit for the three switches on the control ports.

FIG. 10K shows a circuit for the display module.

FIG. 10L shows a circuit for low voltage detection.

FIG. 10M shows a circuit for the display driver chip program programming interface.

The purpose of the circuits shown in FIGS. 10A-10M and the functions they perform are the following: 1. Receive input signals from the temperature sensor 121; 2. Convert the input signal from the temperature sensor 121 to a temperature for display on the display 117 of the housing 206; 3. Accept user input to set the display for Centigrade or Fahrenheit; 4. Turn the light (LED) on and off based on current temperature and target temperature; 5. Receive user input to adjust the conversion of function 2 based on the type of container for the smoke substrate 207; 6. Receive user input to set a target temperature; and 7. Turn on notification to the user such as sound or light or both to notify the user that the smoke substrate 207 is at the target temperature.

Use of the device 200 with a standard vaporizing rig 300 is shown in FIG. 11. The vaporizing rig 300 has a mouthpiece 302 and a container (banger) 205 having an open top 208. In use, the smoke substrate 207 that generates a vapor when heated is placed into the container (banger) 205. The banger 205 can be translucent or transparent. The smoke substrate 207 is heated to a temperature that generates a vapor, such as with a butane torch. The temperature of the target material 207 is detected by the device 200, and this is affected by moving the extension 220 relative to the housing 206 so the temperature sensor 121 is at a location proximate to the container 205, such as 2 inches or less from the smoke substrate 207. Thus, the height and angle of the temperature sensor 121 relative to the smoke substrate 207 can be changed. Once the desired temperature is reached, the user places the banger cap 204 onto/into the open top 208 of the banger 205, and then the user inhales the vapor through the mouthpiece 302.

Although the prior art device has been described with regard to preferred versions, other versions are possible. For example, the device 200 can be provided with a Bluetooth connection for use with a smart phone. The temperature sensor 121 can communicate wirelessly rather than be wired. Multiple temperature sensors 121 can be provided to determine the temperature of multiple smoke substrates 207. The extension 220 can be removable such as with fasteners (such as screws) so a first extension can be changed out for a second extension of a different length or for a different temperature sensor that is effective in a different temperature range.

The prior art had many advantages, including the following:

The device 200 is hands free, meaning, the housing base 100 is placed on a surface, such as a tabletop or desktop, and the temperature sensor 121 can then be moved, via extension 224, independently of housing 206, into close proximity to the heated container 205 to be sensed. This configuration is ideal because the user does not have to hold the device 200/temperature sensor 121 steady and in place while the device 200 takes temperature readings. If the device 200 were hand-held, the device 200 and temperature sensor 121 would be unstable and subject to undesirable movement caused by the user's hands. Additionally, both of the user's hands are free for dabbing, which is highly desirable.

Along the same lines, the flexible extension 224 allows the temperature sensor 121 to be in the position under the banger/container 205 and still have the device 200 facing the user so the user can view the display 117.

Device 200 has recess 202 for holding a banger cap 204. This is desirable because after use, banger cap 204 is hot and sticky and due to its shape, cannot hold itself upright. Accordingly, recess 202 provides a safe and secure place to hold a hot and sticky banger cap 204 while the banger cap 204 is not in use.

The device 200 also has the ability to adjust the output for the different type of container 205 being used. This is ideal because many users have different types of containers 205 made from different materials and the device 200 can adapt accordingly.

The device 200 can be programmed or adapted to measure the time it takes for the smoke substrate 207 to be heated to reaching the desired temperature and track that over time and when certain parameters are met, the user can be alerted to replace the container 205. This is because the more times a container 205 is heated, the container 205 loses the ability to transmit heat to the target material.

The device 200 can also track how many times a smoke substrate 207 (such as a dab) has been heated and the effectiveness of different bangers over time. This would be valuable to an end user to see which brand of banger works best and to also adjust the way they prepare the banger prior to dabbing.

### Smoking Substrates

Smoking substrates 207, such as tobacco in cigarettes and cigars, cannabis in joints, scented chemicals on wicks, and the like burn or vaporize during use to create smoke. For example, cigars and cigarettes are conventional smoking articles that have tobacco, and when burned tobacco smoke is created; while joints are conventional smoking articles that contain cannabis in some countries and states, and when burned cannabis smoke is created.

Some plants, plant byproducts, plant parts, natural oils, synthetic oils, and medicinal drugs collectively, “smoke substrates”) 207, as shown at FIG. 12, are (a) capable of being heated and vaporized 11 so the resulting smoke 12 can be inhaled, smelled, tasted, and/or ingested by a user 14; and (b) grown throughout the world. It is also recognized that smoke substrates 207—for example and not limited to tobacco, cannabis, hashish, natural oils, opium, hash, and/or prescription drugs—can be (i) grown at a particular farm and/or region; and (ii) manufactured in a particular industrial plant and/or region. That particular farm, plant, industrial plant, and/or region, according to smoking substrate experts, generates smoke 12 that has (a) distinct smoke properties and (b) medical benefits.

Applicant does not claim to be a “smoking substrate expert” but has developed a method, system, and tool that capitalizes on those smoke substrate experts' opinions. The method, system, and tool are designed to maximize the smoke substrate's alleged smoke properties and medical benefits to a user.

### Best Temperature Index (BTI)

Smoke substrate experts assert that each specific smoke substrate 207, from a farm, a plant, an industrial plant, and/or region, heats and vaporizes (thermal energy) 11 best at (a) a specific temperature range or (b) a specific temperature—collectively referred to as a Best Temperature Index (BTI) 300—to obtain the optimum smoke effects—medicinal benefits or alternative properties. Whether that is true, is not relevant for this application.

### How Smoke Products Burn

It is understood smoke products 207, like tobacco burn at temperatures ranging from approximately 600° C. to over 900° C., depending on the plant, the plant variety, how the plant was processed after harvesting, and ambient conditions.

The hottest temperatures, around 800° C. to 1,100° C., occur at the tip of a cigarette or cigar during a puff, wherein oxygen causes rapid oxidation to primarily create CO₂ and water.

Immediately behind the tip is called the pyrolysis zone. The pyrolysis zone (a) has lower temperatures, such as 200° C. to 600° C.; (b) is oxygen deprived, wherein the heat breaks down tobacco into volatile flavor compounds (aroma, oils, sugars) without full combustion, forming the aerosol that becomes smoke 12.

Behind the pyrolysis zone is the condensation zone. The condensation zone has even cooler temperatures that cause some heavier tars and compounds from the smoke 12 vapor to condense and get filtered by the unburnt tobacco 10.

It is also understood that when organic matter is heated at increasing temperatures in open containers, the following processes generally occur, in successive or overlapping stages:

Below about 100° C., volatiles, including some water, evaporate. Heat-sensitive substances, such as vitamins and proteins, may partially change or decompose at this stage.

At about 100° C. or slightly higher, any remaining water that is merely absorbed in the material is driven off. And some solid substances, like fats, waxes, and sugars, may melt and separate.

Between 100 and 500° C., many common organic molecules break down. Most sugars start decomposing at 160-180° C. Cellulose, a major component of wood, paper, and cotton fabrics, decomposes at about 350° C. Lignin, another major wood component, starts decomposing at about 350° C., but continues releasing volatile products up to 500° C. The decomposition products usually include water, carbon monoxide and/or carbon dioxide, as well as a large number of organic compounds. Gases and volatile products leave the sample, and some of the gases and volatile products may condense again as smoke. Some volatile products may ignite and burn, creating a visible flame. The non-volatile residues typically become richer in carbon and form large, disordered molecules, with colors ranging between brown and black. At this point the matter is said to have been “carbonized”.

At 200-300° C., if oxygen is in the chemical reaction, the carbonaceous residue may start to burn, in a highly exothermic reaction, often with no or little visible flame. Once carbon combustion starts, the temperature rises spontaneously, turning the residue into a glowing ember and releasing carbon dioxide and/or monoxide. At this stage, some of the nitrogen still remaining in the residue may be oxidized into nitrogen oxides like NO₂, N₂O. Sulfur and other elements like chlorine and arsenic may be oxidized and volatilized at this stage.

Once combustion of the carbonaceous residue is complete, a powdery or solid mineral residue (commonly called ash) is often left behind, consisting of inorganic oxidized materials of high melting point. Some of the ash may have left during combustion, entrained by the gases as fly ash or particulate emissions. Metals present in the original matter usually remain in the ash as oxides or carbonates, such as potash.

### SUMMARY OF THE INVENTION

A device, a system, and method of using a smoke substrate that is authentic and used at the best temperature index to maximize the alleged smoke substrate medicinal and/or alternative benefits.

### BRIEF DESCRIPTION OF THE FIGURES

FIG. 1 (prior art) is a perspective view of a temperature sensing device according to the present invention, with a banger held in a receiving recess of the device;

FIG. 2 (prior art) is a front elevation view of the device of FIG. 1;

FIG. 3 (prior art) is a top plan view of the device of FIG. 1;

FIG. 4 (prior art) is a left side elevation view of the device of FIG. 1;

FIG. 5 (prior art) is a sectional view of the device of FIG. 1 taken along line 5-5 of FIG. 1;

FIG. 6 (prior art) is a right side elevation view of the device of FIG. 1;

FIG. 7 (prior art) is a sectional view of the device of FIG. 1, taken along line 7-7 of FIG. 6;

FIG. 8 (prior art) is an exploded view showing the components of the device of FIG. 1;

FIG. 9 (prior art) is a flow chart showing the programing by a user of the device of FIG. 1;

FIGS. 10A-10M (prior art) schematically show circuit diagrams for the circuits used in the device of FIG. 1;

FIG. 11 (prior art) is a perspective view of use of the device of FIG. 1 with a vaporizing rig;

FIG. 12 (prior art) is overview of smoke substrate being heated and inhaled by a user;

FIG. 13 illustrates a schematic of determining a best temperature index for a smoke substrate, authenticity of the smoke product, and using the BTI and a digital platform;

FIG. 14 illustrates the exterior of the smoke generating device and the thermal conducting device; and

FIG. 15 illustrates the interior of the smoke generating device.

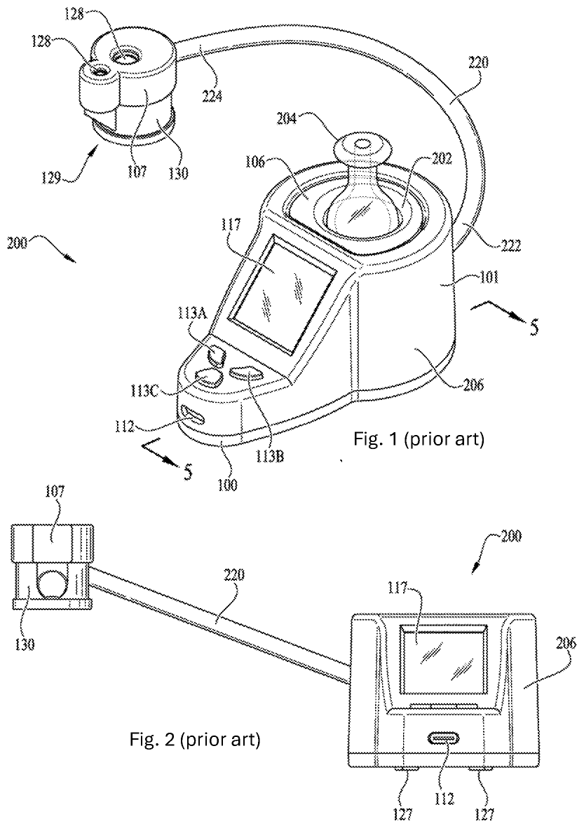

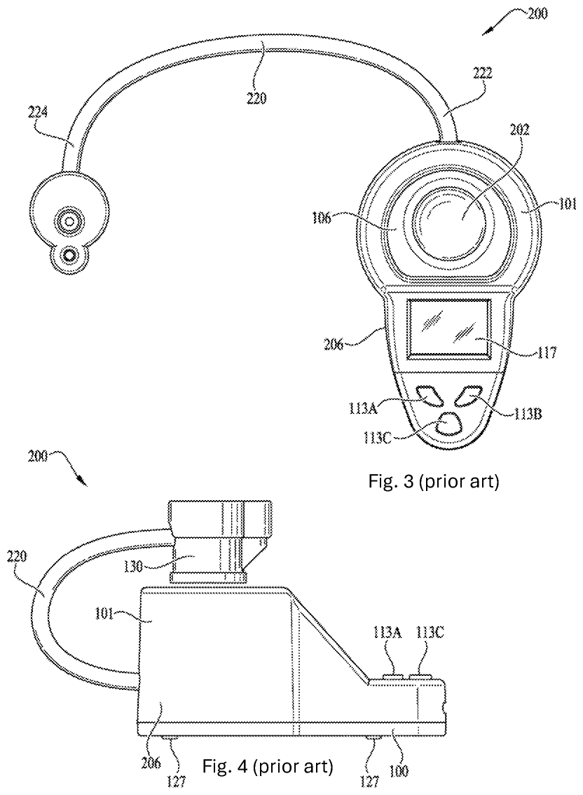

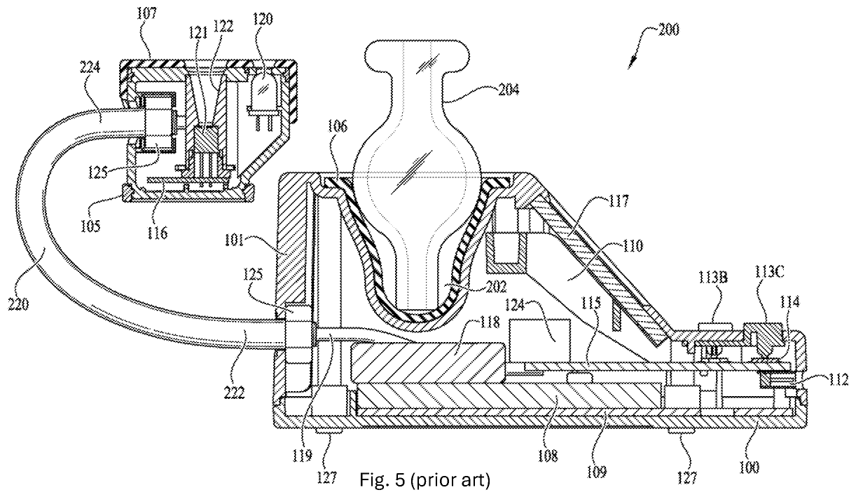

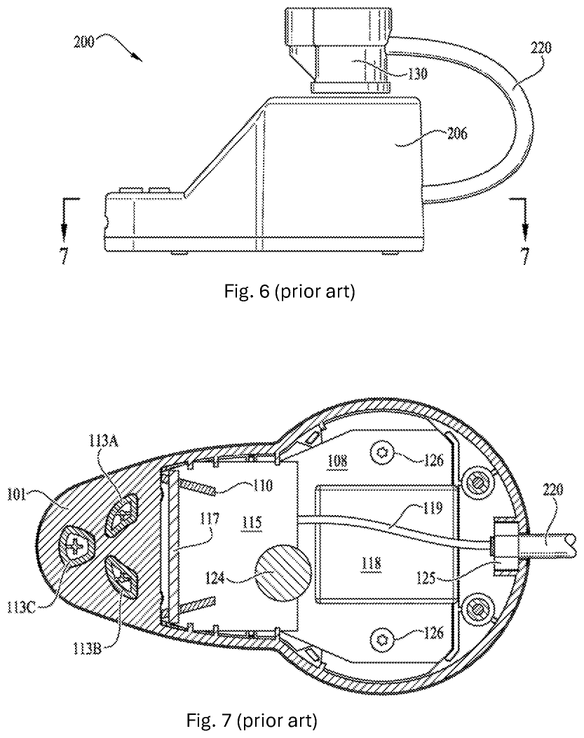

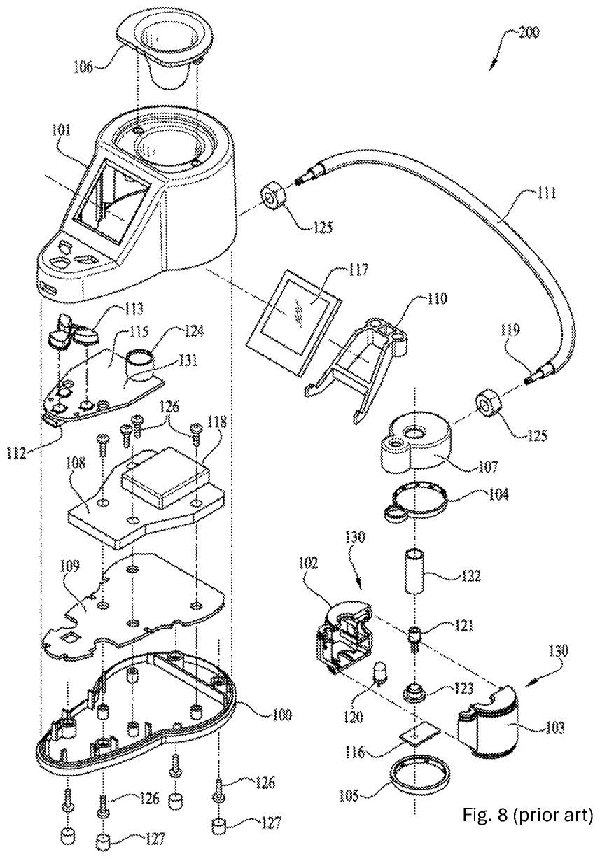

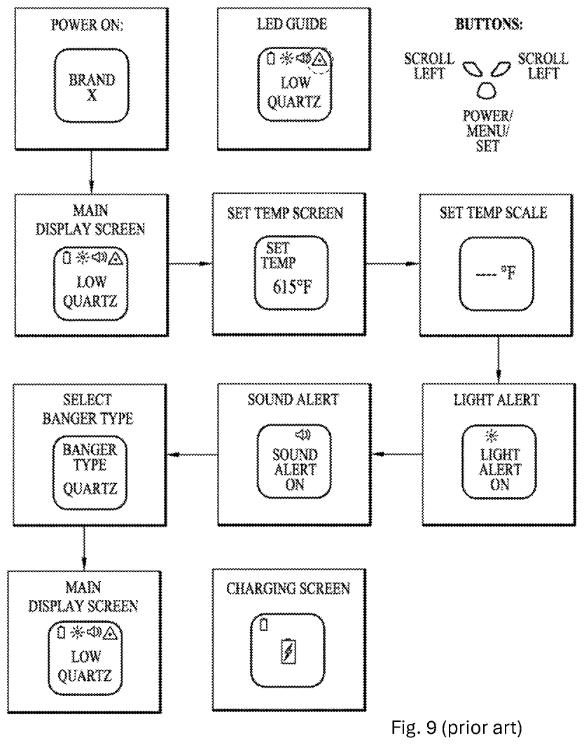

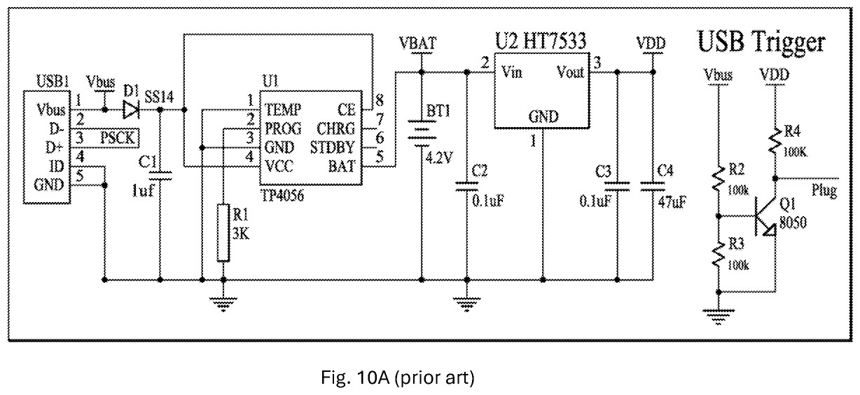

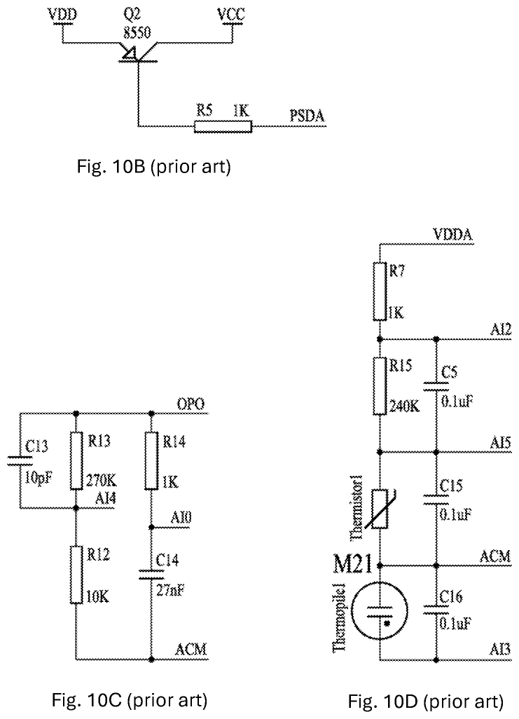

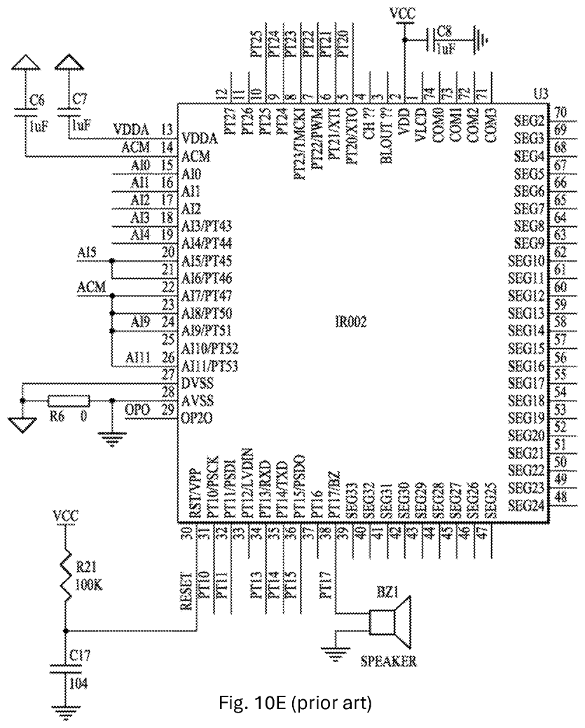

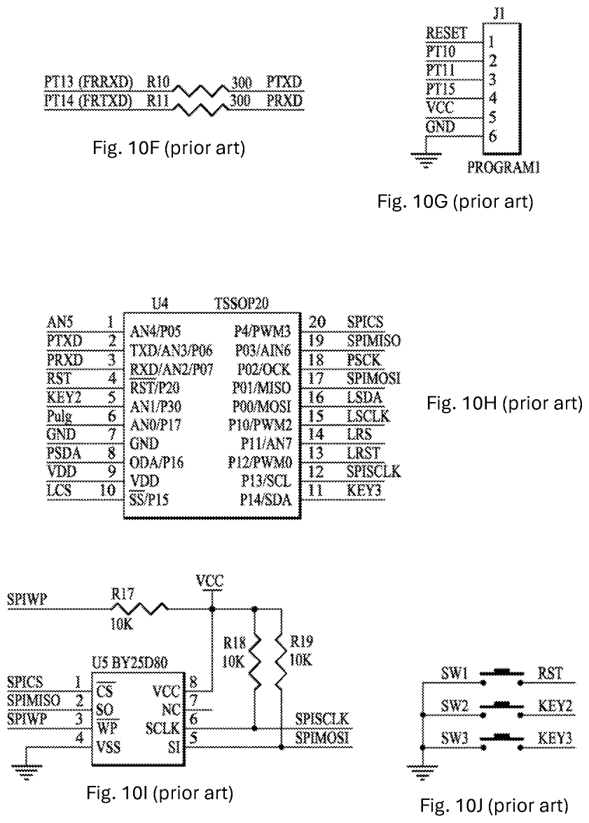

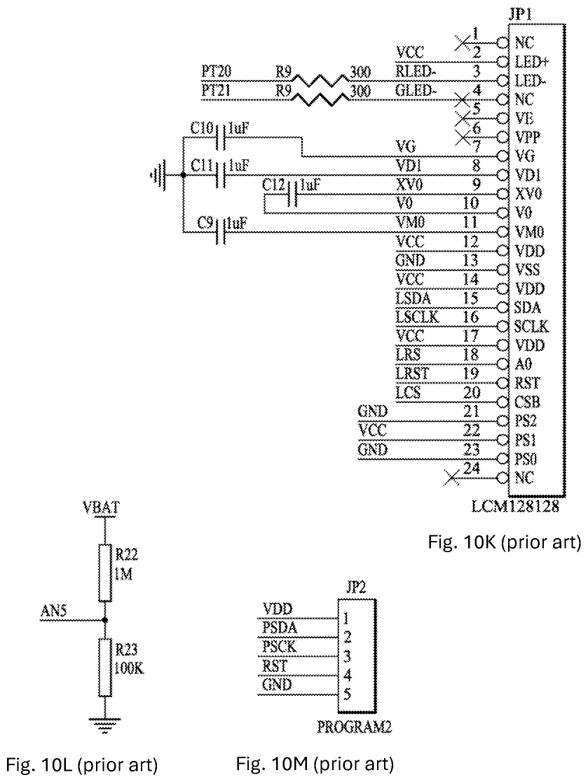

### DETAILED DESCRIPTION OF THE INVENTION

To maximize the smoke substrate's 207 alleged smoke properties and medical benefits, Applicant created a method, a system, and a device that allows a user or third party to (a) identify or confirm the BTI 300 for each specific smoke substrate 207 and (b) utilize the BTI 300 for each specific smoke substrate 207 to obtain the optimum smoke effects 12—medicinal or alternative purposes.

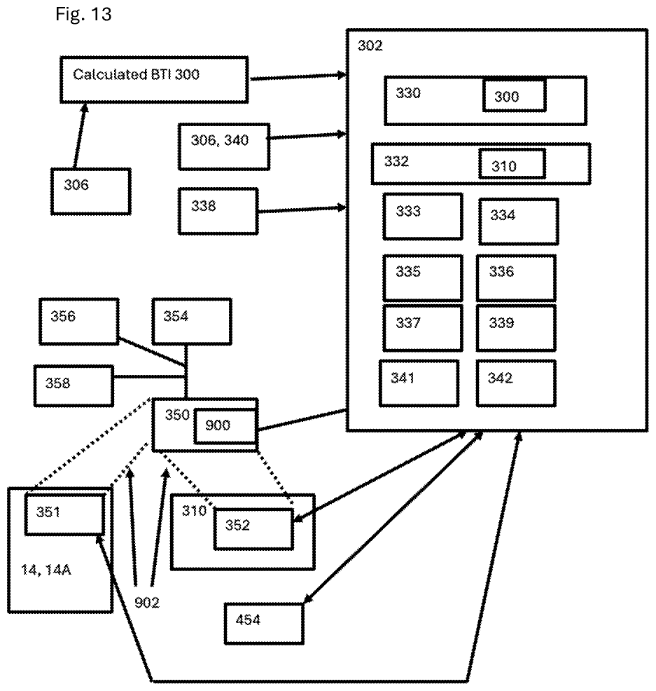

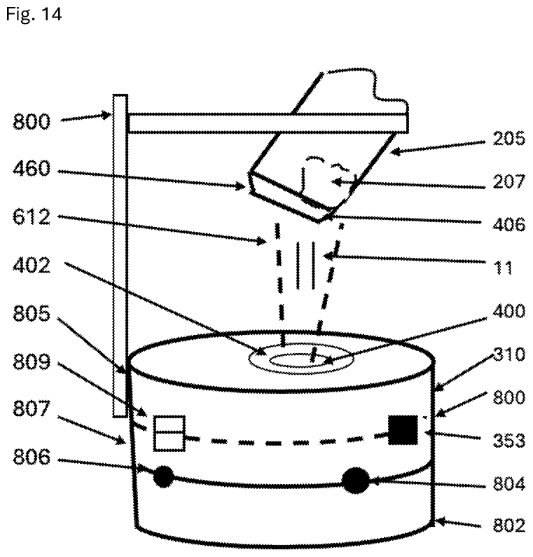

Once the BTI 300 is calculated or determined, the BTI 300 for each specific smoke substrate 207 can be recorded onto a computer database system or a digital platform 302 as shown at FIG. 13. After the BTI 300 is recorded on the computer database 302, the smoke product user 14 or smoke product user's health care provider (collectively “initiator 14a”) can position the specific smoke substrate 207 into the thermal conducting device 205 (see, FIG. 14) so a smoke generating device 310 heats and vaporizes the specific smoke substrate 207 at or near the BTI 300 in order to optimize and/or maximize the specific smoke substrate's 207 smoke 12—medicinal or alternative purposes—and (b) the thermal conducting device 205 directs the smoke 12 toward the user 14.

That way, the initiator 14a can confidently heat and vaporize the smoke substrate 207 at or near its BTI 300 so the user 14 can obtain optimum medicinal or alternative effects.

It is understood that as of December 2025; U.S. federal law doesn't allow certain smoke substrates 207, like marijuana or cocaine, to be smoked or vaporized. Despite the U.S. Federal government's rules, many U.S. states and foreign countries allow medical use of smoke substrates 207 to treat pain, nausea, and other medical symptoms. Moreover, some states and foreign countries permit recreational marijuana use. And some foreign countries permit use of other smoke products, like (a) stimulants such as cocaine, methamphetamine, 3,4-Methylenedioxymethamphetamine (ecstasy); (b) depressants like heroin, opioids, barbiturates; (c) hallucinogens like LSD and psilocybin mushrooms; and (d) cannabis products that have both hallucinogenic and depressant properties.

One known cannabis product is called medical marijuana; medical marijuana comes from a plant called Cannabis sativa. It is understood that medical marijuana is used to allegedly ease symptoms caused by certain medical conditions, whether that is true depends on which expert is being relied upon. Medical marijuana contains chemicals called active compounds. The active compounds are presumed, by some medical practitioners, to ease certain symptoms. Known active compounds include and are not limited to delta-9 tetrahydrocannabinol (THC)—a known psychoactive compound—and cannabidiol (CBD)—a nonintoxicating compound.

THC presumably acts on the brain and affects mood, behavior, and thoughts, called psychoactive effects. U.S. federal law does not allow the use of whole plant Cannabis sativa or its parts for any purpose.

That said, CBD from the hemp plant is legal under federal law when the hemp product contains less than 0.3% THC.

For purposes of this application, the term smoke substrate 207 refers to what is currently legal under (a) US federal law, for this US patent application, since it is expected that US federal law regarding smoke substrates 207 will be amended, and (b) the respective foreign country's law, for foreign patent application(s), since it is uncertain at the time this application is being filed what are the specific laws for each foreign country that this application will be filed.

It is also understood the US Food and Drug Administration has approved a few medicines that contain low-grade THC or CBD. Those medicines, which include and are not limited to the trademarked drugs of Alkem Laboratories Limited's Marinol® brand antiemetic, Wellhouse Pharma's Syndros® brand pharmaceutical preparation for the treatment of anorexia associated with weight loss in patients with acquired immunodeficiency syndrome, and nausea and vomiting associated with cancer chemotherapy and Bausch health US, LLC's Cesamet® brand antiemetic. Those medications require a prescription and may ease chemotherapy-related nausea, stimulating the appetite of patients with debilitating illnesses like H.I.V./AIDS, and easing some pediatric seizure disorders. Thus, the term smoke substrate 207 can have numerous definitions depending on the jurisdiction.

### Platform

The BTI 300 is the temperature or temperature range that heats and vaporizes (vaporizes means “to change from a liquid or solid into a gas or vapor”) a particular smoke substrate 207 to generate smoke 12 that provides the optimum medicinal or alternative effects to the user 14. The BTI 300 for each smoke substrate 207 can be disclosed, as shown at FIG. 13, on the digital platform 302 by at least one of numerous sources 306. The at least one of numerous sources 306 can also provide information about the particular smoke substrate 207.

Information, provided by the at least one of numerous sources 306, about the particular smoke substrate 207 may be recorded or disclosed on the digital platform 302. That smoke product information can include
(A) smoke substrate 207 information 330 includes (i) product name, (ii) strain and/or genetic information, (iii) location or region where grown or made, (iv) ingredients, (v) ingredients' concentration, (vi) harvest date, (vii) processing date, (viii) duration of curing period, (ix) sell-by date, (x) expiration date, (xi) BTI 300, (xii) flower, and (xiii) variations thereof; and
(B) a smoke generating device recommendation 332 that suggests which smoke generating device 310 for the disclosed smoke substrate 207 that can effectively apply the BTI 300 to the smoke substrate 207 to obtain the optimum smoke effects—medicinal or alternative purposes.

Other information disclosed on the digital platform 302 can include (1) advertisements 333, (2) testimonial videos and/or statements 334 such as reviews about the smoking substrate's strain, genetic information, BTI 300, and recommended smoke generating device 310, (4) instructions 335, for example, on how to use the recommended smoke generating device 310, and (5) podcasts 336 about the smoke substrates, smoke generating devices, BTIs, and how to confirm the authenticity of smoke substrates from scanning QR codes 350 and confirming the same with the digital platform 302.

The digital platform 302 permits users 14 to compare, on a computer or a mobile application, different smoke substrate products based on strain, type, genetics, and recommended BTI, to assist the user 14 or initiator 14A select the desired smoke substrate. That comparison can also be directed toward identifying smoke substrates that have similar BTIs so users can select different smoke substrates based on the BTI and the smoke generating devices that work well at that BTI.

The digital platform 302 triggers a blockchain-based system—a decentralized, distributed digital ledger technology (DLT) that records transactions and data across a network of computers, ensuring high security, transparency, and immutability without a central authority wherein data is stored in cryptographically linked blocks, making alteration nearly impossible—in the information 300, and a quick response (QR) code 350 (explained in greater detail below), integrated within a computer or mobile application 351 that verifies the authenticity of the smoking substrate 207, and guarantees the users 14 (or initiator 14A) receives accurate and trustworthy information 300 about the smoking substrate 207 from the digital platform 302 and on the QR code 350. That authenticity can be performed by (a) the QR reader 352 on the smoke generating device 310 through a window 353 reading 902 a blockchain value 900 on the QR code 350 and corresponding with (i) the digital platform 302 to confirm the authenticity of the smoking substrate 207, and/or (ii) a microprocessor 454 (defined below) that interconnects, normally through conventional wireless technology, for example and not limited to Bluetooth® wireless communications, to the digital platform 302 to confirm the authenticity of the smoking substrate 207; or (b) the computer or mobile application 351 on a respective computer or mobile computer system, like a cell phone or pad, that has its QR reader that is used by the user 14 or initiator 14A wherein the QR reader from the computer or mobile computer system reads 902 the blockchain value 900 on the QR code 350 and corresponds with the digital platform 302 to confirm the authenticity of the smoking substrate 207.

In addition, the digital platform 302 can have (a) a correspondence sub-platform 337 that permits users 14, initiators 14a, and sources 306 to communicate—video and/or written—about (a) the smoke substrate 207, (b) the smoke product's 207 effects, (c) the smoke substrate's 207 BTI 300 and whether the BTI 300 should be maintained or adjusted, (d) history with the smoke product 207, (e) the provider's respective smoking history with the smoking substrate 207, and (f) the smoke's 12 effects. The correspondence sub-platform 337 could permit (a) the general public to provide, review, and edit the correspondence, (b) select members of the general public to provide and edit the correspondence while the remaining members of the general public can only read and/or review the correspondence, or (c) select members of the general public can provide, review, and edit the correspondence. The parameters of who can provide, edit, and review any information on the digital platform 302 can be controlled by an administrator 338 of the digital platform 302 using conventional administrative tools to control who, what, where, when, and how the correspondence sub-platform 337 and the digital platform 302 can be accessed and/or edited (authorization protocols).

As expressed above, the digital platform 302 can have the correspondence sub-platform 338 permit industry smoke product leaders (when authorized—depends on the digital platform's authorization protocol) or users (when authorized—depends on the digital platform's authorization protocol) to comment and/or review the smoke substrate 207—on a live stream event and/or a pre-recorded event—to discuss their thoughts and reviews about the smoke substrate 207. Also on the correspondence sub-platform 337, the user 14 and initiator 14A can receive advertisements 333 about new, current, and/or old smoke substrates 207, smoke generating devices 310, smoke substrate accessories, and other non-smoke substrate goods and/or services. In addition, the user 14 and initiator 14A can identify the user's favorite 339 smoke substrates 207 on the digital platform 302. The administrator 338 of the digital platform 302 can permit smoke substrate entities 306, 340 to (a) pay for advertising 333 and (b) disclose “new release” information that can be monetized by selling these marketing components to a smoke substrate growers, smoke substrate makers, smoke substrate manufacturers, smoke substrate brand, smoke substrate retailer, smoke substrate distributor, and equivalents thereof. The smoke substrate entities include and are not limited to growers of smoke substrates, curers of smoke substrates, manufacturers of smoke substrates and/or smoke generating devices, brand owners of smoke substrates and/or smoke generating devices, retailers of smoke substrates and/or smoke generating devices, distributors of smoke substrates and/or smoke generating devices, combinations thereof, and equivalents thereof.

The digital platform can also generate a geotargeted content delivery system within the computer and/or mobile application 351, that permits brands to promote smoke substrate products and updates to users 14 and initiators 14A in specific regions, enhancing marketing.

The smoke substrate entities 306, 340 can have their own portal 341 on the digital platform 302 to add information about their smoke substrate 207, smoke generating devices 310, and other products and services. That way, the digital platform 302 can have content managed exclusively by the smoke product entities 306, 340.

Examples of the numerous sources 306 that provide the BTI 300 for each strain of smoke substrate 207 and the recommended smoke generating device 310 include and are not limited to:
(i) smoke substrate entities 340,
(ii) users 14 and initiators 14A, and/or
(iii) scientists or a scientific based entity that use actual scientific methods, state-of-the-art testing equipment, and/or protocols to measure and record the BTI 300 for smoke substrate strains to generate smoke that optimizes its medicinal or alternative effects through the recommended 332 smoke generating device 310 wherein the data for the BTI 300 and smoke generating device 310 can be repeatedly duplicated and not based on bald assertions.

The information 330 about the smoke product 310 in particular the strain and the BTI 300, and the recommended 332 smoke generating device 310 is recorded onto the digital platform 302 through at least one source 306, and in particular the smoke product entities 340.

The digital platform 302 permits the source 306 to enter its smoke substrate information 330 through a conventional data entry system like a keyboard or an on-screen keyboard, for example, that can appear on a cell phone or pad. When the source 306 is the smoke substrate entity 340, the information disclosed on the digital platform 302 about the smoke substrate 207 could be (A) the smoke substrate's (a) name, (b) strain, (c) ingredients, (d) ingredient concentrations, (e) harvest date, (d) curing dates, (e) temperature while curing, (f) humidity while curing, (g) recommended smoke generating device 310, (h) strain's genetics, and/or (i) the BTI 300; (B) identity of who or which entity received the smoke substrate 207 for processing and/or curing; and/or (C) the brand name that the smoke substrate 207 will be sold under. And if the source 306 is a testing laboratory, the information 330 disclosed on the digital platform 302 about the smoke substrate 207 could be the smoke substrate's BTI 300 with a particular smoke generating device 310.

Once smoke substrate's 207 information 330 is on the digital platform 302 regarding the smoke product's (a) name and strain, and (b) BTI 300 with or without a recommendation 332 for a particular smoke generating device 310, then the digital platform 302 generates the quick response (QR) code 350 through a QR code generator 342. The QR code generator 342 creates the scannable QR code 350 based on the smoke substrate's 207 information 330 provided by at least one of the numerous sources 306 for each particular smoke substrate 207. It is well known that the scannable QR code 350 allows instant access to the information 330 on the digital platform 302 via a smartphone or other conventional devices 351 having a QR code reader 352 used by the user 14 or initiator 14A; or the QR reader 352 on the smoke generating device 310.

The scannable or the QR code 350 generated by the QR code generator 342 can be (a) a static QR code that cannot be altered; (b) a dynamic QR code wherein any source 306 can alter the information 330 on the digital platform 302 for the smoke substrate 207; or (c) a semi-dynamic QR code wherein only sources 306 authorized by (i) an original source 306 of the smoke substrate's information 330 and/or (ii) administrator 338 can alter the information on the digital platform 302 for each particular smoke substrate 207.

The QR code 350 for each smoke substrate 207 can be displayed on the smoke substrate's 207 packaging 354 (preferred because then the QR code is applicable for that specific packaged smoke substrate 207 material), display 356, digital media sites 358, or equivalents thereof.

The user 14 and/or initiator 14A can (a) access the smoke substrate's 207 information 330 from the digital platform 302 on a read-only basis, if the administrator 338 of the digital platform 302 designates the digital platform's information 330 read-only to users 14 and initiators 14A; (b) access and edit the smoke substrate's 207 information 330 on the digital platform 302 if the administrator 338 of the digital platform 302 designates the digital platform's information 302 dynamic to users 14 and initiators 14A; or (c) access as a read-only to all portions of the smoke substrate's 270 information 330 on the digital platform 302 and edit select portions of the smoke substrate's 207 information 330 on the digital platform 302 if the administrator 338 of the digital platform 302 designates the digital platform's information 330 semi-dynamic to users 14 and initiators 14A.

The user 14 and/or initiator 14A can also access the digital platform's 302 smoke substrate 207 information 330 if the user 14 and/or initiator 14A scan the QR code 350, and/or, if possible, read the smoke substrate 207 information 330 on the particular smoke substrate's 207 packaging 354, display cards 356, and/or digital media site 358.

As previously expressed, the information about each smoke substrate 207, each smoke substrate's BTI 300, and the recommended 332 smoke generating device 310 can be (a) printed on the particular smoke substrate's packaging, website, social media site, and/or marketing material and/or (b) accessed through each smoke substrate's 207 respective QR Code 350 or equivalent thereof (for example, a bar code) that could be on the particular smoke substrate's packaging, website, social media site, and/or marketing material.

The smoke substrate's 207 digital platform 302 information 330 can inform the user 14 or initiator 14A about everything disclosed on the digital platform 302 includes and is not limited to (a) BTI 300, (b) the recommended smoke generating device 332, and, (c) optionally, provide (i) marketing information about the particular smoke substrate 207, (ii) description of the particular smoke substrate's chemicals and/or concentration of chemicals; (iii) details about the smoke substrate's flower, and/or (iv) a listing of products containing the particular smoke substrate.

As indicated above, the digital platform 302 can have a “review” section, where smoke product entities 340 (authorized or not—depends on the digital platform's authorization protocol) or users 14 (authorized or not—depends on the digital platform's authorization protocol) can review the smoke substrate—on a live stream event and/or a pre-recorded event—to discuss their thoughts and reviews about particular smoke substrates 207.

The digital platform 302 may also be a paid service that generates revenue through featuring those reviews to promote smoke product entities 340 and equivalents thereof.

### Smoke Generating Device

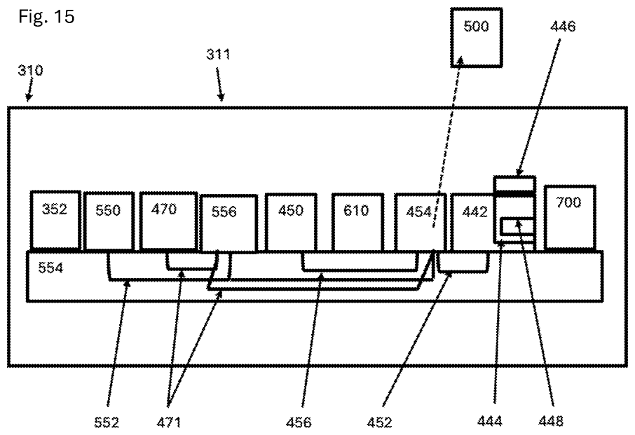

The smoke generating device 310, unlike the prior art, is a self-contained device as shown in FIGS. 14 and 15. It is made of a material that can handle the thermal energy 11 directed toward the thermal conducting device 205 and has areas that could be translucent or transparent so light coming from the smoke generating device 310 can be directed toward the thermal conducting device 205. Examples of the material can be metal, ceramic, glass, thermal polymeric materials like thermoplastics and thermosets, stone, marble, or combinations thereof. The smoke generating device 310 has a heat aperture 400 and an optionally a light space 402 positioned about or near the heat aperture 400. The light space 402 is a translucent or transparent material that permits light from the smoke generating device 310 define or identify an area 406—by a circle, a plurality of lines, a plurality of dots that number 2 or more, or combinations thereof—on the thermal conducting device 205 that should receive the maximum thermal energy 11 radiating through the heat aperture 400 of the smoke generating device 310. The light space 402 can be circular as shown in FIG. 14, polygonic, a plurality of dots, or a combination thereof.

The smoke generating device 310 has a temperature control device 442. The temperature control device 442 measures the temperature radiating from the area 406 on the thermal conducting device 205 where the smoke substrate 207 is supposed to be positioned in the thermal conducting device 205.

The smoke generating device 310 also has a heating element 450. The heating element 450 is capable of generating and directing thermal energy 11 toward the area 406 on the thermal conducting device 205 where the smoke substrate 207 is supposed to be positioned in the thermal conducting device 205.

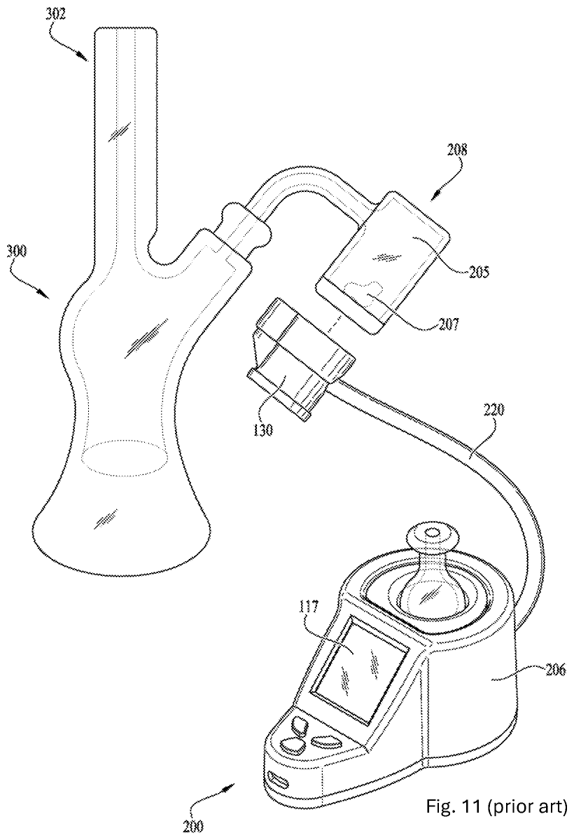

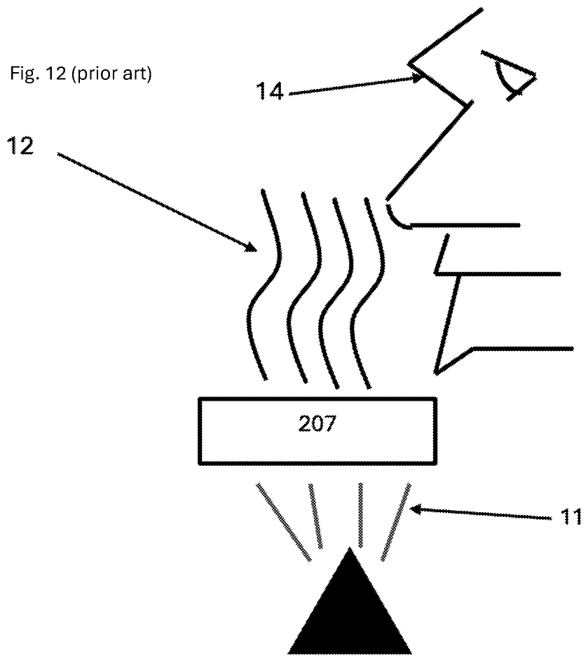

The thermal conducting device 205 has a closed proximal end 460 to contain the smoke substrate 207 and an open end to direct the smoke 12 toward the user 14 (see, FIG. 11. Examples of conventional thermal conducting devices 205 include and are not limited to the container, a test-tube, a beaker, a conical flask (Erlenmeyer flask), a boiling flask (Florence flask), domeless nails, a round bottom flask, a Fernbach flask, a fleaker, a chemex coffeemaker, a banger, a volumetric flask, a cell culture flask, a filtering flask, a recover flask, a distilling flask, a freeze drying flask, a Kjeldhl flask, and equivalents thereof.

The thermal conducting device 205 is capable of receiving thermal energy 11 from the heating element 450 when the thermal conducting device 205 is properly positioned to receive the thermal energy 11 generated by the heating element 450. Depending on the heating element 450 selected for the smoke generating device 310, the heating element 450 can operate through conduction, convection, and/or radiation.

To diminish problems, conduction is heat transfer through direct contact, where vibrating molecules bump into adjacent molecules, passing energy along like the thermal conducting device 205 directly contacting the heating element 450. Convection is heat transfer through the movement of fluids (liquids or gases), creating currents as warmer, less dense fluid rises and cooler, denser fluid sinks, like the thermal conducting device 205 positioned in, for example, boiling water, or near a radiator. Radiation is heat transfer via electromagnetic waves (like infrared), for example, the thermal conducting device 205 is positioned near a flame or a conventional infrared heater.

When the thermal conducting device 205 containing the smoke substrate 207 is heated by the heating element 450, the heating element 450 is designed to heat the smoke substrate 207 to the BTI 300 so the smoke substrate 207 heats and vaporizes to generate the desired smoke 12 that has the medicinal or alternative effects that could be inhaled, smelled, ingested, and/or tasted by the user 14. Once the smoke substrate 207 reaches the BTI 300, the smoke generating device 310 generates a sound through the microprocessor 454. The microprocessor 454 can have a Pulse Width Modulation (PWM), built-in Digital-to-Analog Converters (DACs), or external sound generator chips. The simplest methods involve driving a piezo speaker or small speaker directly via a PWM for simple tones, while more complex audio requires dedicated sound chips like Texas Instrument's SN76489 or General Instrument's AY-3-8910 for waveforms, melodies, and sound effects. The sound informs the user 14 or initiator 14A when the smoke 12 should be generating its alleged maximized medicinal effects.

Controlling the heating element's 450 temperature to obtain and hopefully maintain the BTI 300 occurs through
(A) electronic circuitry, for example and not limited to:
   (i) voltage control,
   (ii) pulse width modulation, and/or
   (iii) solid state relays.

It is understood that a specific temperature range that is within 3.0 degrees Celsius, preferably within 1.5 degrees Celsius, or more preferably within 0.3 degrees Celsius of the BTI is acceptable since conventional heating elements 450 and thermostats 444 are not always 100% accurate.
(B) a human and/or an artificial entity can move or remove the smoke substrate 207 or the thermal conducting device 205 containing the smoke substrate 207 away from thermal energy 11 generated by the heating element 450, in response to bells, alarms and whistles like that disclosed in the prior art is ineffective and potentially deleterious to the user 14 or the initiator 14a. Obviously, this option is not recommended.
(C) monitor the thermal energy radiating from the area 406 of the thermal conducting device 205 containing the smoke substrate 207 so the heating element 450 can be (a) automatically adjusted to generate less or more thermal energy 11, or (b) maintained to provide the current thermal energy 11; or
(E) combinations thereof.

To accomplish those objectives, (a) the temperature control device 442 has at least one temperature sensor 444 that (a) measures the temperature radiating from the area 406 of the thermal conducting device 205 containing a smoke substrate 207 and (b) transmits an area temperature signal 452 corresponding to the measured temperature to a microprocessor, microcontroller, a proportional-integral-derivative controller, or Arduino-based system 454.

When the microprocessor 454 recognizes the area temperature signal 452 is below, at, or exceeds the BTI 300, then the microprocessor 454 transmits a control signal 456 to the heating element 450, normally through a solid state relay or field effect transistor (normally a MOSFET), to decrease, maintain, or increase thermal energy 11 provided by the heating element 450 to the area 406.

That way, the thermal energy 11 from the heating element 450 heats and vaporizes the smoke substrate 207 at or near the BTI 300 to generate smoke 12 having its alleged maximized medicinal effects. This occurs automatically without having a user 14 or initiator 14A move the thermal conducting device 205 or adjust the heating element 450, as required in the prior art.

The thermal conducting device 205 has a temperature above absolute zero (−273.15° C. or 0 Kelvin) and as a result emits electromagnetic radiation from its surface. That electromagnetic radiation is proportional to the thermal conducting device's 205 natural temperature. The thermal conducting device's 205 electromagnetic radiation may have various electromagnetic waves that are measurable upon an electromagnetic radiation spectrum. And a part of that spectrum that is relevant for measuring the thermal conducting device's 205 temperature from a non-contact temperature measuring device 444 is infrared radiation since the thermal conducting device's 205 infrared radiation is emitted into the thermal conducting device's 205 surrounding atmosphere.

The temperature sensor 444 has a lens 446, as shown in FIG. 15, that focuses electromagnetic radiation from the thermal conducting device's 205, in particular the area 406, onto a temperature detector element 448. The temperature detector element 448 generates the area temperature signal 452 that is proportional to the electromagnetic radiation focused on the temperature detector element 448, from the thermal conducting device 205, and more in particular, the area 406.

The area temperature signal 452 is amplified and, using conventional digital signal processing techniques—mathematically manipulate digitized real-world signals (like audio, images) using algorithms for analysis, enhancement, or compression, relying on core concepts like sampling, quantization, and digital filtering (FIR/IIR); key methods include the Fast Fourier Transform (FFT) for frequency analysis, convolution for filtering, and Z-transforms for system analysis, enabling applications from noise cancellation and speech recognition to image processing in modern electronics—in microprocessor 454 so the area temperature signal 452 is transformed into the control signal 456.

The area temperature signal 452 can be shown on a display 500 or transmitted, through conventional wireless technology—using conventional Bluetooth®, Wi-Fi®, NFC®, or Zigbee® equipment—, as an analog output signal onto a computer display, hand-held (for example, a phone) and/or wrist-based (for example, a watch) display 351.

Advantages of using a non-contact temperature measurement device are that the non-contact temperature measurement device can provide (1) temperature measurements of (a) a moving thermal conducting device 207, (b) a static thermal conducting device 205, and/or (c) an overheated thermal conducting device 205, (2) non-destructive measurement, (3) non-interactive measurements that have no influence on the measured thermal conducting device 205, (4) measurement point durability, and (5) little to no mechanical wear. At the same time, the disadvantages are that the non-contact temperature measurement device's measurements may not be accurate since (a) the non-contact temperature measurement device does not contact the thermal conducting device 205, and (b) the temperature measurement is based on electromagnetic radiation waves coming from the thermal conducting device 205.

The electromagnetic spectrum is a mixture of different electromagnetic waves, and each electromagnetic wave has a distinct wavelength or frequency. The electromagnetic radiation spectrum covers a wavelength area of about 23 decimal powers and varies from sector to sector in origin, creation, and application of the radiation. All electromagnetic radiation follows similar principles of diffraction, refraction, reflection, and polarization. The electromagnetic radiation's expansion speed corresponds to the light speed under normal conditions; the result of multiplying wavelength with frequency is constant (λf=c).

Infrared radiation covers a very limited part in the whole range of the electromagnetic spectrum since it starts at the visible range of about 0.78 μm and ends at wavelengths of approximately 1000 μm. Wavelengths ranging from 0.7 μm to 14 μm are important for infrared temperature measurement. Above those wavelengths the energy level is so low, that the non-contact temperature measurement device is not sensitive enough to detect the infrared waves of 15 μm or greater.

A black body is an “idealized physical body that absorbs all incident electromagnetic radiation regardless of frequency or angle of incidence. And the radiation emitted by a black body in thermal equilibrium with its environment is called black-body radiation.” The black body has neither reflective nor transmissive properties and radiates the maximum energy possible at each wavelength. The concentration of the radiation does not depend on angles. The black body is the basis for understanding the physical principles of non-contact temperature measurement and for calibrating infrared thermometers.

The black body has a thermal hollow body with a small hole at one end. When the black body is heated and reaches a certain temperature, and there is a temperature equilibrium inside the hollow body, then the black-body emits black-body radiation of the certain temperature from the hole. And for each temperature range and application purpose the construction of these black bodies depends on material and the geometric structure. If the hole is very small compared to the surface as a whole, the interference of an ideal state is very small.

A contact & non-contact temperature and non-contact distance sensors device that measures an object's temperature is disclosed.

There are two-types of temperature sensors—contact types and non-contact types. The contact types include thermocouples, thermistors, and resistance thermometers. The non-contact temperature sensors include infrared temperature sensors and color temperature sensors.

### Contact Type Temperature Measurement Devices

A thermocouple is an electrical device consisting of two dissimilar electrical conductors forming an electrical junction. The thermocouple produces a temperature-dependent voltage because of the well-known Seebeck effect, and that voltage can be interpreted to measure temperature. Thermocouples have at least one limitation, and that limitation is accuracy since system errors of less than one degree Celsius (° C.) can be difficult to achieve with a thermocouple.

A thermistor is a semiconductor type of a resistor whose resistance is strongly dependent on temperature, more so than in standard resistors.

Thermistors differ from resistance temperature detectors (RTDs) in that the material used in a thermistor is generally a ceramic or polymer, while RTDs use pure metals. The temperature response is also different; RTDs are useful over larger temperature ranges—for example coiled RTDs have a temperature range from 0° C. to 850° C.; while thermistors typically achieve a greater precision within a limited temperature range, typically −90° C. to 130° C.

In this invention, a contact-type temperature sensor 550 measures the ambient air's temperature; and transmits an ambient temperature signal 552 that corresponds to the measured ambient air temperature measurement through a printed circuit board 554 to a memory unit 556, positioned on, as shown in FIG. 15, the printed circuit board 554, and/or the microprocessor 454.

The microprocessor 454 receives the ambient air temperature signal 552 and further modifies the area temperature signal 452 to adjust the area's measured temperature to account for the ambient air temperature prior to transmitting the control signal 456.

### Non-Contact Type Temperature Measurement Devices

The temperature detector element 448 can be an infrared temperature sensor that can be made to have compact sensor heads, wherein the temperature detecting distance can be 1 mm to 3 meters or greater. The infrared temperature sensor 448 should be installed below the aperture 400.

The temperature control device 442 measures the thermal conducting device's 207, preferably, the area's 406 temperature. As identified above, the temperature control device 442 converts the measured temperatures of the thermal conducting device 205 into electric signals 452 that can be modified by the ambient temperature measurement and can optionally output the temperature measurements onto a display screen 500 or equivalent thereof.

Infrared temperature sensors detect the thermal conducting device's 205 emitted infrared rays to measure the thermal conducting device's 205 temperature by:

The lens 446 collecting and directing the thermal energy emitted as infrared rays from the thermal conducting device's 205, in particular from the area 406, to the sensing element 448 or sometimes referred to as a thermopile.

The thermopile 448 absorbs the infrared rays which, in this embodiment, raises the thermopile's temperature, and generates the area temperature signal 452 according to the recorded temperature of the thermal conducting device 205; and

The electric signals 452 is amplified and corrected to generate the output signal 456 based on the thermal conducting device's 205 emissivity—the ease of thermal radiation (infrared rays) emitting from the thermal conducting device 205 wherein air has an emissivity value of 0 because all infrared rays reflect or pass through air while the thermal conducting device 205 could have a maximum, yet not really, emissivity value of 1. That said, it is understood that most objects have an emissivity value between 0 and 1. The output signal 456 can be displayed 500 or reported.

The above-identified thermopile's structure can be numerous thermocouples connected in series. The thermocouple's hot junctions are positioned in the center while the thermocouple's cold junctions are positioned along the periphery. The infrared rays collected by the lens 446 strikes the hot junctions, so the hot junctions are preferably heated. The Seebeck effect—the phenomenon where a thermoelectromotive force is generated when two conductors of different metals are electrically connected to form a closed circuit and a temperature difference is applied to both ends—generates a voltage difference between the hot and cold junctions, thereby allowing for the temperature measurement.

Infrared temperature sensors measure the strength of radiant heat to determine the temperature. There are three types of infrared temperature sensors; which are as follows:
Total radiation temperature sensors, wherein the total sensors measure temperatures from the integral value within the entire wavelength range;
Wide-band radiation temperature sensors, wherein the wide-band sensors measure temperatures by using only wavelengths; and
One-color temperature sensors, wherein the one-color sensors measure temperatures based on the strength of the radiant heat at one wavelength.

The infrared temperature sensors cannot measure the temperature of the inside of a target or of gases. Emissivity also must be set in accordance with the target.

In contrast, two-color temperature sensors measure radiant heat by using two different wavelengths and determining the temperature from the ratio between the radiances. The two-color temperature sensors are characterized by fewer errors even when measuring targets that are smaller than the spot diameter of the sensor. The two-color temperature sensors have the disadvantage of unstable temperature values in locations with dust or water vapor in the air or when the target is measured through a dirty window, because of the diffusion of radiant heat.

The non-contact-type temperature control device 442 measures thermal energy coming from the thermal conducting device's 205, preferably, the area's 406 temperature. The area 406 is where the smoke substrate 207 is positioned or is believed to be positioned at or near the closed proximal end 460. That temperature-measured area 406 can be visually confirmed by (a) a multi-colored/programmable LED ring, (b) a solid-colored LED ring, (c) one or a plurality of LED dot lights (for example 3 dots to form a triangle, 4 dots to form a square, et al.) or (d) a plurality of laser diodes that can appears on the thermal conducting device 205. Each of those lighting arrangements can be provided by a specific lighting system 610. The lighting system 610 is aligned with the non-contact-type temperature control device 442 so the non-contact-type temperature control device 442, heating element 450, and lighting systems 610 are, respectively, directed to measure the temperature of the area 406, heat the area 406, and highlight the area 406 to make sure all three components are properly aligned to the area 406. The lighting system 610 transmits a light beam, light ring, and/or a series of lighted beams to form a LED/Laser cone/field 612—onto the area 406 where the smoke substrate 207 is positioned or is believed to be positioned at the closed proximal end 460.

### Distance Measurement

The smoke generating device 310 can also include a distance measurement unit 470. A common distance measurement unit 470 is an ultrasonic distance sensor, a laser distance sensor, and/or an infrared distance sensor. A conventional ultrasonic sensor measures the distance from the smoke generating device's 310 top surface 311 to the area 406. That distance measurement occurs by ultrasonic sound waves. Similarly, a conventional infrared sensor can measure the same distance using infrared waves at a frequency that does not interfere with the infrared waves used by the non-contact-type temperature sensor 442. The infrared waves range from 1,000 μm to 0.7 μm; wherein far infrared waves range from 1,000 μm to 15 μm; while thermal infrared ranges from 15 μm to 8 μm, mid infrared ranges from 8 μm to 3 μm, and near infrared ranges from 3 μm to 0.7 μm. The conventional infrared distance sensor 470 measures distance based on triangulated reflected infrared light. For example, a conventional infrared LED (wherein the distance sensor 470 could also be a part of the lighting systems 610) emits an infrared light beam that reflects off a targeted object and is picked up by a receiver or other photosensitive element.

A conventional laser distance sensor 470—sometimes called a laser displacement sensor—have various means to operate. One common method involves time-of-flight wherein the laser distance sensor 470 calculates the distance from the time it takes light to reflect. A second common method involves triangulation wherein the distance measurement is calculated by analyzing the angle of the reflected laser beam.

A conventional ultrasonic distance sensor 470 uses a transducer to send and receive ultrasonic pulses that relay back information about an object's proximity.

High-frequency sound waves reflect across boundaries to produce distinct echo patterns.

Ultrasonic sensors work by sending out a sound wave at a frequency above the range of human hearing. The transducer of the sensor acts as a microphone to receive and send the ultrasonic sound. The ultrasonic sensor, normally, uses a single transducer to send a pulse and to receive the echo. The sensor determines the distance to a target by measuring time lapses between the sending and receiving of the ultrasonic pulse.

The working principle of this module is simple. It sends an ultrasonic pulse out, for example, above 20 kHz, which travels through the air, and if there is an obstacle or object like the thermal conducting device's 205 area 406 in front of the top surface 311, then the ultrasonic pulse will bounce back to the distance sensor 470. By calculating the travel time and the speed of sound, the distance between the top surface 311 to the thermal conductive device's 205 area 406 can be calculated.

The distance measurement unit 470 (a) is positioned to measure the distance between the top surface 311 to the thermal conductive device's 205 area 406 and (b) transmits a distance electrical signal 471 that corresponds to the measured distance through the printed circuit board to the memory unit 556 and/or the microprocessor 454.

The microprocessor 454 receives the ambient temperature measurement 552, the temperature signal 452, and the distance electrical signal 471; and calculates the temperature of the thermal conductive device's 205 area 406.

The smoke generating device 310 has a top section 800 and a bottom section 802. The top section 800 is capable of pivoting along a x-axis 804 along, for example, a x-axis shaft, a y-axis 806 along, for example, a y-axis shaft, or a combination of both the x and y-axes through, for example, a 2-axis pan/tilt gimbal device so the top section 800 can be easily positioned toward the thermal conductive device's 205 area 406. In addition, the top section 800 can have an upper section 805 and a lower section 807 that are interconnected by a hinge 809 to permit a user 14 or initiator 14A to, for example, alter the power source for the smoke generating device 310.

The smoke generating device 310 top surface 311 has (a) at least one cavity 400 that exposes the distance measurement unit 470, the non-contact type measurement unit 442, the heating element 450, the contact type measurement unit 550, and the desired light system 610, optional, to highlight the thermal conductive device's 207 area 406. Positioned below the top surface 311 is the printed circuit board 554. The printed circuit board 554 electronically interconnects to each of the following units: the distance measurement unit 470, the non-contact type measurement unit 442, the heating element 450, the contact type measurement unit 550, and the desired light system 610, central processing unit 454 and/or a memory unit 556, an optional, display unit 500 that is capable of conveying the ambient temperature, the measured temperature at the thermal conductive device's 205 area 406, the distance between the top surface 311 and the thermal conductive device's 205 area 406, and/or the calculated actual hottest temperature of the thermal conductive device's 205 area 406; a QR reader/receiver 700 and a power source (not shown).

The power source can be a battery unit, for example a lithium-ion polymer battery within or outside the thermal conducting device 205, and/or a conventional electrical connection that connects to a conventional power source, for example, an electrical outlet or computer that can provide the necessary power to the thermal conducting device 205 to operate. The lithium-ion polymer battery can be recharged through a charging pad path from a conventional wireless charging pad and/or a conventional wireless charging plate, or through a conventional interconnection to an electrical outlet.

The central processing unit 454 and/or memory unit 556 (collectively the calculating unit) is interconnected to the distance measurement unit 470, the non-contact type measurement unit 442, the heating element 450, the contact type measurement unit 550, and the desired light system 610, central processing unit 454 and/or a memory unit 556, an optional, display unit 500 that is capable of conveying the ambient temperature, the measured temperature at the thermal conductive device's 205 area 406, the distance between the top surface 311 and the thermal conductive device's 205 area 406, and/or the calculated actual hottest temperature of the thermal conductive device's 205 area 406; the QR reader/receiver 700, and a power source (not shown).

If the smoke generating device 310 has an optional stand 800, wherein the stand 800 ensures the thermal conducting device's 205 area 406 is a fixed distance from the smoke generating device 310. The distance measurement unit 470 is optional when the stand 800 is used. That means, the calculated actual hottest temperature of the thermal conductive device's 205 area 406 is determined with a standard value for the distance between the thermal conducting device's 205 area 406 and the smoke generating device 310.

Likewise, the contact type measurement unit 550 is optional when the smoke generating device 310 operates in a standard room temperature 18° C. to 25° C. environment. That means, the calculated actual hottest temperature of the thermal conductive device's 205 area 406 is determined with a standard value for the ambient air temperature measurement.

The smoke generating device 310 does receive operating instructions. The operating instructions can be received through numerous means. The preferred operating instructions are received by the smoke generating device 310 through the QR reader 700 capable of reading each smoke substrate's 207 QR Code 350. Once the QR reader 700 reads the smoke substrate's 207 QR Code 350, the QR reader 700 transmits the information to the central processing unit 554 and/or memory unit 556. The central processing unit 554 and/or memory unit 556 is able to interpret the information from the QR Code 350 to operate the smoke generating device 310 to achieve the BTI 300 for the smoke substrate 207. Alternatively, the central processing unit 554 and/or memory unit 556 is able to communicate, through conventional wireless technology, with the digital platform 302 to obtain the instructions to operate the smoke generating device 310 to achieve the BTI 300 for the smoke substrate 207.

To accomplish those objectives, the smoke generating device's 310 QR code reader 700 reads or obtains the smoke substrate's 207 BTI 300 from the smoke substrate's QR code 350 and/or the digital platform 302, and conveys the smoke substrate's information to the central processing unit 554 and/or memory unit 556. Once the BTI 300 is obtained, the temperature control device 442 measures the thermal energy coming from the thermal conducting device's 205 area 406 containing the smoke substrate 205 and depending on the actual temperature measurement 456 the central processing unit 554 alters, adjusts, or maintains the thermal energy 11 coming from the heating element 450 so the actual thermal energy from the thermal conducting device's 207 area 406 approaches, or is at the BTI 300; and if the actual thermal energy from the thermal conducting device's 207 area 406 exceeds the BTI 300 then the central processing unit 554 decreases the thermal energy generated from the heating element 450 that is directed toward the thermal conducting device's 207 area 406. And when the smoke substrate 207 is at or near the BTI, then the smoke substrate 207 generates smoke 12 having its alleged maximized medicinal effects.

If desired, the smoke generating device 310 could transmit, through a wire or wirelessly, a signal to the display unit 500 for the user 14, or initiator 14A to review the actual temperature applied to the thermal conducting device's 207 area 406 to obtain the smoking substrate's alleged maximized medicinal effects. The display unit 500 could be a stand-alone screen, a screen attached to the smoke generating device 310, a computer, tablet, or smart-phone wirelessly interconnected to the smoke generating device 310 through conventional wireless technology.

The operating instructions can also be received by the central processing unit 454 and/or memory unit 556 through (a) a computer program and/or (b) conventional manual controls, for example, switches, buttons, and other conventional devices to adjust the on, off, and, if possible, intensity of each component in the smoke generating device 310.

The operating instructions can also be pre-programmed to control the operation of the light system, the distance measurement unit; the non-contact type measurement unit; and the contact type measurement unit. The processing unit is capable of being interconnected to a program input device like a remote computer unit. The program input device permits the software program to be edited or changed.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

1. A method of heating a smoke substrate; comprising:
(a) permitting a party to enter a best temperature index (BTI) for the smoke substrate onto a digital platform;
(b) the digital platform generating a quick response code (QR code) based on information about the smoke substrate and corresponding BTI;
(c) in either order:
(c.i) inserting the smoke substrate into a thermal conducting device so the smoke substrate is positioned in an expected area of the thermal conducting device; and
(c.ii) a smoke generating device
(i) obtaining the information about the smoke substrate and corresponding BTI by reading the QR code through a QR code reader or accessing the digital platform through a wireless connection with a user's phone or computer, and transferring that information to a microprocessor or memory chip;
(ii) positioning and adjusting the smoke generating device so a lighting system in the smoke generating device can project light on to the thermal conducting device to define the area;
(ii) applying thermal energy generated by a heating element in the smoke generating device toward the area,
(iii) measuring the thermal energy from the area through a non-contact temperature sensor in the smoke generating device, and transmitting the measured area's thermal energy temperature to the microprocessor;
(iv) measuring the distance from the smoke generating device's top surface to the area through a distance sensor, and transmitting the measured distance to the microprocessor;
(v) measuring the ambient temperature around the smoke generating device through a contact temperature sensor, and transmitting the measured ambient temperature to the microprocessor;
(vi) calculating an actual temperature applied to the smoke substrate through the measured area's thermal energy temperature, the measured ambient temperature, the measured distance, and the average emissivity of the thermal conducting device, and comparing the actual temperature to the BTI;
(vii) adjusting or maintaining the thermal energy generated by the heating element so the smoking substrate is at or near the BTI to generate smoke having maximized medicinal benefits or alternative properties.

2. The method of claim 1 further comprising positioning the thermal conducting device onto a stand so the area is a predetermined distance from the smoke generating device.

3. The method of claim 1 further positioning an aperture in the smoke generating device so the thermal energy from the heating element, the lighting system, and the non-contact temperature sensor are directed to the area.

4. The method of claim 1 wherein the QR code has a blockchain code and the QR reader (a) reads the QR code to obtain the blockchain code to confirm the smoke substrate is an authentic product and/or (b) corresponds with the digital platform to confirm the smoke substrate is an authentic product.

5. The method of claim 1 wherein the QR code has a blockchain code and the QR reader (a) reads the QR code to transmit the blockchain code to the microprocessor, the microprocessor confirms the smoke substrate is an authentic product and/or (b) corresponds with the digital platform to confirm the smoke substrate is an authentic product.

6. The method of claim 1 wherein the smoke substrate is selected from the group consisting of tobacco, cannabis, hashish, natural oils, opium, hash, and prescription drugs.

7. A smoke generating device comprising:
a quick response (QR) reader to obtain information about a smoke substrate and best temperature index (BTI) for the smoke substrate through a QR code, or a microprocessor that wirelessly interconnects to a digital platform that generates the QR code after the smoke substrate's information and BTI is recorded in the digital platform,
the microprocessor receives the smoke substrate information and BTI;
a lighting system, the lighting system projects light on to a thermal conducting device to define an area that should have the smoking substrate in the area;
a heating element that generates thermal energy and directs the thermal area toward the area;
a non-contact temperature sensor, the non-contact temperature sensor measures the thermal energy from the area, and transmits the measured area's thermal energy temperature to the microprocessor;
a distance sensor, the distance sensor measures the distance from the smoke generating device's top surface to the area and transmits the measured distance to the microprocessor;
a contact temperature sensor, the contact temperature sensor measures the ambient temperature around the smoke generating device, and transmits the measured ambient temperature to the microprocessor;
the microprocessor (a) calculates an actual temperature applied to the smoke substrate through the measured area's thermal energy temperature, the measured ambient temperature, the measured distance, and an average emissivity of the thermal conducting device, (b) compares the actual temperature to the BTI, and (c) adjusts or maintains the thermal energy generated by the heating element so the smoking substrate is at or near the BTI to generate smoke having maximized medicinal benefits or alternative properties.

8. The device of claim 7 further comprising positioning the thermal conducting device onto a stand so the area is a predetermined distance from the smoke generating device.

9. The device of claim 7 further positioning an aperture in the smoke generating device so the thermal energy from the heating element, the lighting system, and the non-contact temperature sensor are directed to the area.

10. The device of claim 7 wherein the QR code has a blockchain code and the QR reader (a) reads the QR code to obtain the blockchain code to confirm the smoke substrate is an authentic product and/or (b) corresponds with the digital platform to confirm the smoke substrate is an authentic product.

11. The device of claim 7 wherein the QR code has a blockchain code and the QR reader (a) reads the QR code to transmit the blockchain code to the microprocessor, the microprocessor confirms the smoke substrate is an authentic product and/or (b) corresponds with the digital platform to confirm the smoke substrate is an authentic product.

12. The device of claim 7 wherein the smoke substrate is selected from the group consisting of tobacco, cannabis, hashish, natural oils, opium, hash, and prescription drugs.

13. A system of heating a smoke substrate; comprising:
(a) permitting a party to enter a best temperature index (BTI) for the smoke substrate onto a digital platform;
(b) the digital platform generating a quick response code (QR code) based on information about the smoke substrate and corresponding BTI;
(c) in either order:
(c.i) inserting the smoke substrate into a thermal conducting device so the smoke substrate is positioned in an expected area of the thermal conducting device; and
(c.ii) a smoke generating device
(i) obtaining the information about the smoke substrate and corresponding BTI by reading the QR code through a QR code reader or accessing the digital platform through a wireless connection with a user's phone or computer, and transferring that information to a microprocessor or memory chip;
(ii) positioning and adjusting the smoke generating device so a lighting system in the smoke generating device can project light on to the thermal conducting device to define the area;
(ii) applying thermal energy generated by a heating element in the smoke generating device toward the area,
(iii) measuring the thermal energy from the area through a non-contact temperature sensor in the smoke generating device, and transmitting the measured area's thermal energy temperature to the microprocessor;
(iv) measuring the distance from the smoke generating device's top surface to the area through a distance sensor, and transmitting the measured distance to the microprocessor;
(v) measuring the ambient temperature around the smoke generating device through a contact temperature sensor, and transmitting the measured ambient temperature to the microprocessor;
(vi) calculating an actual temperature applied to the smoke substrate through the measured area's thermal energy temperature, the measured ambient temperature, the measured distance, and the average emissivity of the thermal conducting device, and comparing the actual temperature to the BTI;
(vii) adjusting or maintaining the thermal energy generated by the heating element so the smoking substrate is at or near the BTI to generate smoke having maximized medicinal benefits or alternative properties.

14. The system of claim 13 further comprising positioning the thermal conducting device onto a stand so the area is a predetermined distance from the smoke generating device.

15. The system of claim 13 further positioning an aperture in the smoke generating device so the thermal energy from the heating element, the lighting system, and the non-contact temperature sensor are directed to the area.

16. The system of claim 13 wherein the QR code has a blockchain code and the QR reader (a) reads the QR code to obtain the blockchain code to confirm the smoke substrate is an authentic product and/or (b) corresponds with the digital platform to confirm the smoke substrate is an authentic product.

17. The system of claim 13 wherein the QR code has a blockchain code and the QR reader (a) reads the QR code to transmit the blockchain code to the microprocessor, the microprocessor confirms the smoke substrate is an authentic product and/or (b) corresponds with the digital platform to confirm the smoke substrate is an authentic product.

18. The system of claim 13 wherein the smoke substrate is selected from the group consisting of tobacco, cannabis, hashish, natural oils, opium, hash, and prescription drugs.
